(19) United States
(12) Patent Application Publication
(10) Pub. No.: US 2022/0411828 A1
(43) Pub. Date: Dec. 29, 2022

(54) METHOD FOR BIOLOGICAL IN-SITU METHANATION OF CO2 AND H2 IN A BIOREACTOR

(71) Applicant: VERBIO Vereinigte BioEnergie AG, Zörbig (DE)

(72) Inventors: Fabian Bonk, Leipzig (DE); Jan Köckeritz, Halle (DE); Niklas Böttger, Radegast (DE); Michael Schlimbach, Halle (Saale) (DE); Oliver Lüdtke, Markkleeberg (DE)

(73) Assignee: VERBIO Vereinigte BioEnergie AG, Zörbig (DE)

(21) Appl. No.: 17/831,534

(22) Filed: Jun. 3, 2022

(30) Foreign Application Priority Data
Jun. 23, 2021 (EP) 21181020.5

(51) Int. Cl. C12P 5/02; C12M 1/107; C12M 1/00; C12M 1/34

(52) U.S. Cl. CPC C12P 5/023; C12M 21/04; C12M 47/18; C12M 41/26

(57) ABSTRACT

The invention relates to a method for the biological in-situ methanation of CO₂ and H₂ in a bioreactor. The method includes feeding an organic substrate into the bioreactor wherein at least part of the organic substrate is converted to a biogas comprising methane and carbon dioxide by means of microorganisms. The organic substrate includes crude fiber and at least 0.15 kg of crude fiber per m³ bioreactor volume per day is fed into to the bioreactor. The bioreactor is operated at between about 20-45° C. H₂ is fed to the CO₂ into the bioreactor to produce methane.

### The invention relates to a method for the biological in-situ methanation of CO₂ and H₂ in a bioreactor.

### CROSS-REFERENCE TO RELATED APPLICATIONS

This application claims the benefit of foreign priority to European Patent Application No. EP21181020.5, filed Jun. 23, 2021, which is incorporated by reference in its entirety for all purposes.

### TECHNOLOGICAL BACKGROUND

The conversion (methanation) of carbon dioxide (CO₂) and hydrogen (H₂) to methane (CH₄) can be catalytic, usually nickel-based, or biological, i.e., by means of microorganisms. In both cases, methanation takes place after the following overall reaction:

CO₂+4H₂→CH₄+2H₂O (1)

In the case of biological conversion, CO₂ and H₂ are also used by the microorganisms involved for their anabolism, e.g., for the synthesis of enzymes. However, the conversion of CO₂ and H₂ in the anabolism is low compared to the conversion to methane. Biological methanation has the advantage that it is less susceptible to catalyst toxins such as hydrogen sulfide (H₂S), which can be present in CO₂ flows from biogas plants, for example.

The biological methanation of CO₂ and H₂ is divided into the two concepts ex-situ and in-situ. In the in-situ concept, H₂ is fed to a bioreactor in addition to organic substrate. The organic substrate is fermented to CH₄ and CO₂, among others. This CO₂ is in turn converted to CH₄ using H₂. In the ex-situ concept, only CO₂ and H₂ is converted in the bioreactor, but no organic substrate. The main advantage of the in-situ concept is that existing bioreactors, which have so far been fed exclusively with organic substrate, can be used, whereas an ex-situ concept requires the cost-intensive construction of new bioreactors.

Using the in-situ concept, CH₄ production rates from fed H₂ of 0.016 cubic meters of CH₄ per cubic meter of bioreactor volume per hour (m³/m³/h) could be achieved under thermophilic temperature control (approx. 55° C.) (EP2771472B1, Example 1, Table 3). However, in the in-situ method with thermophilic temperature control taught in patent EP2771472B1, the feeding of acidic substrate is necessary. This is disadvantageous, since acid substrates are not available everywhere and thus have to be transported to the plant over long distances, entailing high costs. Moreover, the embodiments also suggest that the method can only be used for a low organic loading rate of less than 1.7 kg of organic dry substance (VS) in the form of organic substrate per m³ of bioreactor volume per day. The use of such low organic loading rates is also rendered obvious since it is known that processes with thermophilic temperature control exhibit low process stability.

The problem of low process stability with thermophilic temperature control is addressed in patent EP2586868B1 by the fact that H₂ is only used in an existing bioreactor cascade. In this cascade, the first bioreactor is fed with organic substrate, but not with H₂. The effluent from the first bioreactor is fed to a second bioreactor that, in turn, is loaded with H₂. The second bioreactor is only exposed to a small amount of organic substrate and thus to a low organic loading rate, since a large part of the organic substrate has already been fermented in the first bioreactor. However, the disadvantages are that a bioreactor cascade must be available in the first place, i.e., this method cannot be applied to single-stage plants, and the first bioreactor of such a cascade is not available for H₂ conversion.

In addition, a fundamental problem with thermophilic temperature control is the high energy input required to maintain the elevated temperature. Using an in-situ concept for mesophilic temperature control (approx. 37° C.) is therefore preferable.

However, the in-situ concept under mesophilic temperature control has so far only achieved CH₄ production rates from CO₂ and H₂ of about 0.0017 cubic meters of CH₄ under standard conditions (273.15 K and 1.01325 bar) per m³ bioreactor volume per hour (Nm³/m³/h) (DE102012112889A1, [0075]). Excess sludge from a sewage treatment plant with an VS content between 2.2 to 2.8% was fed as organic substrate. Despite mesophilic temperature control, an organic loading rate of only 1.25 kg VS/m³/d was achieved.

The invention disclosed in claim 1 is based on the problem that, in the current state of the art in a bioreactor with the in-situ concept under mesophilic temperature control and with high organic loading rate of organic substrate, it was not possible to increase the CH₄ production rate sufficiently by feeding H₂.

### SUMMARY

The invention relates to a method for the biological in-situ methanation of CO₂ and H₂ in a bioreactor. The method comprises feeding an organic substrate to the bioreactor wherein at least part of the organic substrate is converted to biogas by means of microorganisms, wherein the organic substrate includes crude fiber and the organic substrate is fed into the bioreactor at a rate where it includes at least 0.15 kg of crude fiber per m³ bioreactor volume per day, and operating the bioreactor is at 20-45° C. H₂ is fed to the bioreactor and at least part of the H₂ together with CO₂ is converted to methane by means of microorganisms.

Surprisingly, it was found that even bioreactors with high organic loading rate have high potential to convert H₂. However, high CH₄ production rates from fed H₂ are only achievable with a high supply of crude fiber. This is unlike the typical operation of biogas reactors as, normally, crude fibers, which consist mainly of biomass that is difficult to degrade, should be avoided in order to prevent a high and difficult-to-handle viscosity from developing, as well as insufficient utilization of the available bioreactor volume. Surprisingly, however, feeding a high amount of crude fiber has been shown to be helpful with respect to an increased CH₄ production rate from fed H₂. This is based, without any claim to completeness and with the restriction to a purely theoretical approach, on the increase of the retention time of hydrogen bubbles, as well as on the increase of the culture surface of microorganisms.

### DETAILED DESCRIPTION

### Definitions

In the context of the present invention, the term “in-situ” refers to the biological methanation of fed CO₂ and H₂ in a bioreactor in which organic substrate is also converted to biogas.

In the context of the present invention, the dry substance (TS) refers to the solid residue obtained after removing the solvent (e.g., water) from a suspension (e.g., from a stillage) or from a solution. That is, the solid residue refers to the totality of all previously dissolved or suspended solids (e.g., crude fibers and salts).

In the context of the present invention, organic dry substance (VS) refers to the part of the TS that does not remain as ash when the TS is incinerated.

In the context of the present invention, the organic loading rate of a bioreactor is defined as the average mass flow of VS fed to the bioreactor within one day, based on the bioreactor volume. This results in the unit kg VS/m³/d.

In the context of the present invention, the term “organic substrate” refers to liquid or solid organic material flows containing VS which are fed to the bioreactor. This includes, but is not limited to, by-products of industries processing biomass, such as stillage from a bioethanol plant or potato pulp from starch production, as well as residues from agriculture and forestry, such as straw, oat husks, rice hulls or sawdust. The term “organic substrate” does not encompass the gases CO₂ and H₂.

In the context of the present invention, the term “microorganisms” refers to an undefined mixed culture of bacteria and/or archaea that may be found in typical biogas plants. “Microorganisms” does not imply a pure culture of a particular species.

In the context of the present invention, the measurand of the steam-volatile ammonium nitrogen (NH₄—N) is used to assess the ammonium content. To determine this, ammonia is expelled from the sample with the aid of steam and collected in a boric acid solution, followed by titration using hydrochloric acid. The concentration of ammonium in the sample can be inferred from the consumption of hydrochloric acid.

In the context of the present invention, the term bioreactor volume refers to the volume of liquid located in the bioreactor. This also refers to all standardized quantities based on the bioreactor volume, such as CH₄ production rate, H₂ feed rate and CO₂ feed rate. This bioreactor volume represents the place where biochemical reactions occur.

In the context of the present invention, hydrogen (H₂) refers to hydrogen in its molecular form.

In the context of the present invention, the H₂ feed rate and the CO₂ feed rate refer to the volume of H₂ or CO₂ under standard conditions (N for short, 273.15 K and 1.01325 bar) in m³ per m³ of bioreactor volume, which is fed to the bioreactor on average every hour. This results in the unit Nm³/m³/h.

In the context of the present invention, the CH₄ production rate refers to the volume of CH₄ under standard conditions (N for short, 273.15 K and 1.01325 bar) in m³ per m³ bioreactor volume, which is formed in the bioreactor on average every hour. This results in the unit Nm³/m³/h. In the present invention, a distinction is made between CH₄ production rate from VS and CH₄ production rate from H₂ fed to the bioreactor. The CH₄ production rate from VS corresponds to the CH₄ production rate produced in the bioreactor without feeding H₂. The CH₄ production rate from fed H₂ is defined in equation 2

$$CH 4 - Production rate from fed H 2 = \frac{H 2 Feed rate - H 2 Output Rate}{4} \qquad 2 )$$

The person skilled in the art is aware that H₂ can be formed in the intermediate steps of the fermentation of VS, acidogenesis and acetogenesis; the H₂ together with CO₂ can be converted to CH₄. This H₂ is not part of the H₂ fed into the bioreactor (H₂ feed rate). Equation (2) neglects the part of hydrogen that is not converted to CH₄ in catabolism by the microorganisms involved in the H₂ conversion but is used in anabolism to synthesize molecules such as enzymes.

In the context of the present invention, the term “crude fiber content” refers to the fraction of an organic substrate referred to as “crude fiber” in food and feed analysis according to Weender. The measuring method for determining the crude fiber content is described in the following source, which is incorporated by reference:

Association of German Agricultural Testing and Research Institutes (VDLUFA [Verband Deutscher Landwirtschaftlicher Untersuchungs—und Forschungsanstalten]) (publisher), 1993: Methode 6.1.1. Bestimmung der Rohfaser (WEENDER-Verfahren) [Method 6.1.1 Determination of crude fiber (WEENDER method)]. Handbuch der Landwirtschaftlichen Versuchs—und Untersuchungsmethodik [Handbook of Agricultural Experimental and Investigative Methodology] (VDLUFA-Methodenbuch [VDLUFA method booklet]), Volume III, Die Untersuchung von Futtermitteln [The examination of food and feed], 3rd Edition, VDLUFA Verlag [Publisher], Darmstadt.

The feeding of crude fiber to the bioreactor is expressed as kg crude fiber/m³/d and represents the mass of crude fiber in kg per m³ bioreactor volume supplied to the bioreactor on average in one day.

In the context of the present invention, the term “product gas” refers to the gas leaving the bioreactor in gaseous form. Product gas may comprise a mixture of gaseous substances, in particular CH₄, CO₂, H₂, H₂S and H₂O vapor. In the context of the present invention, biogas refers to a mixture of gases produced during the anaerobic decomposition of organic substrate and consisting mainly of CH₄ and CO₂.

In the context of the present invention, the term “product gas treatment” refers to the partial separation of gas components from the product gas.

### DESCRIPTION OF THE INVENTION

In the present invention, organic substrate is mainly converted to CH₄ and CO₂ in a bioreactor at temperatures between 20-45° C. At the same time, H₂ is fed into the bioreactor with the result that a biological conversion of CO₂ and H₂ to CH₄ occurs.

Surprisingly, it was found that when using the mesophilic temperature range (20-45° C.) instead of the thermophilic temperature range as disclosed in the prior art (EP2771472B1, EP2586868B1), the advantages outweigh the disadvantages and even higher CH₄ production rates can be achieved from fed H₂. So far, the prior art has suggested that thermophilic temperatures are preferable because it is known that microbial activity is higher at thermophilic temperatures than at mesophilic temperatures. Mesophilic temperatures have the advantage of higher process stability and energy efficiency as less thermal energy needs to be provided.

Moreover, in the present invention, a high amount of crude fiber is fed into the bioreactor. In the prior art, substrates low in crude fiber such as whey (EP2771472B1) or sewage sludge (EP2586868B1) have been preferred. According to Weender's food and feed analysis, crude fiber content is the part of an organic substrate that remains after treatment with dilute acids and alkalis, i.e., it is not easily “digestible.” Feeding substrate with a high crude fiber content into a bioreactor is, therefore, generally regarded as a disadvantage since the poorly digestible components it contains can accumulate in the bioreactor, resulting in a high viscosity that is difficult to handle, thus reducing the usable bioreactor volume. The consequence can be problems with floating layers, stirring and pumping issues.

Surprisingly, with respect to feeding H₂ into a bioreactor, it was found that the advantages of feeding one or more organic substrates with high crude fiber content outweigh the disadvantages described above. While not claiming to be exhaustive and limited to a purely theoretical approach, this is based on increasing the retention time of hydrogen bubbles, which improves their transfer to the reactor liquid and their microbial conversion, as well as on increasing the culture surfaces for microorganisms. Based on experimental results, it was determined that at least 0.15 kg crude fiber/m³/d should be fed to achieve high CH₄ production rates as a result of feeding H₂. For comparison, the prior art in EP2771472B1 recommends the feeding of whey, which contains virtually no crude fiber (source: https://www.ingredients101.com/whey.htm, retrieved Apr. 5, 2021). In a preferred embodiment, the method is designed such that 0.15 kg crude fiber/m³/d, preferably 0.2 kg crude fiber/m³/d, particularly preferably 1.0 kg crude fiber/m³/d is fed into the bioreactor.

As a matter of preference, the method is designed in such a way that a majority of the particles of the organic substrate have a size of 10 mm or less.

Mesophilic microorganisms thrive in a temperature range between 20-45° C. (Schiraldi and De Rosa, 2014). It was found, however, that the optimum process control is within a narrower temperature range. In a preferred embodiment, the method is designed such that the bioreactor is operated at a temperature of 35-45° C., preferably at 36-41° C., particularly preferably at 37-39° C.

The microorganisms in the bioreactor are a mixed culture of bacteria and archaea. Such mixed cultures are known to the person skilled in the art in the context of biogas production. In the process steps hydrolysis and acidogenesis, bacteria convert organic substrate to acetic, propionic and butyric acid, H₂ and CO₂, among others. In acetogenesis, bacteria convert propionic and butyric acid to acetic acid, H₂ and CO₂. In methanogenesis, archaea convert H₂ and CO₂, acetic acid and other methyl components to methane. Depending on the substrate, CO₂ is also formed in the methanogenesis step. Compared to pure cultures, as they are mainly used in ex-situ concepts and where, for instance, only one specific methanogenic species is cultivated in a bioreactor, mixed cultures have the advantage that the bioreactor does not have to be operated sterilely to avoid contamination with other microorganisms.

The person skilled in the art is familiar with how to get a biogas process started. For example, an effluent from an existing biogas plant or animal excrement that already contains a mixed culture of bacteria and archaea is used for inoculation.

In a preferred embodiment, the method is such that the hydraulic retention time (HRT) is at least 18 days, preferably at least 25 days, and particularly preferably at least 35 days.

In a preferred embodiment, the method is designed in such a way that the H₂ feed is at least 0.017 Nm³/m³/h, preferably 0.125 Nm³/m³/h, particularly preferably 0.167 Nm³/m³/h. In alternative methods in the prior art, only 0.015 Nm³/m³/h (DE102012112889, [0075]) has been achieved under mesophilic temperature control and 0.118 Nm³/m³/h under thermophilic temperature control (EP2771472B1, [0102], calculated from 1.7 NI/d and 0.6 L bioreactor volume). Thus, with the help of the described invention, there is a possibility to increase the H₂ supply compared to the current state of the art, thus increasing CH₄ production rates and thereby improving the productivity of bioreactors.

In a preferred embodiment, the method is designed such that the crude fibers are supplied in the form of organic substrate, particularly preferably in the form of organic substrate of plant origin. This enables the use of low-cost organic substrates, e.g., residual materials such as straw.

In a preferred embodiment, the method is designed in such a way that the organic substrate is fed into the bioreactor with an organic loading rate of more than 2.5 kg VS/m³/d, particularly preferably more than 3.0 kg VS/m³/d, despite the feeding of H₂. In the current prior art, H₂ has been fed into a bioreactor only at low organic loading rates, e.g., 2.1 kg VS/m³/d in patent EP2586868A2 or 1.7 kg VS/m³/d in patent EP2771472B1. For patent EP2586868A2, the calculation was based on the stated maximum CH₄ production rate from VS (EP2586868A2, [0034]) and a usual gas yield for sewage sludge of 300 NI/kg VS. The calculation of the organic loading rate from patent EP2771472B1 is based on the specified retention time, reactor size and the specified material data of the organic substrate (EP2771472B1, [0101] and [0102]).

In a preferred embodiment, the method is designed such that the H₂ fed to the bioreactor originates from the electrolysis of water. In a particularly preferred embodiment, the method is designed such that the electricity for the electrolysis was generated from renewable sources. This has the advantage that the CH₄ produced in the bioreactor from the electrolysis H₂ is a sustainable, electricity-based fuel obtained independently of oil and natural gas.

If the fed H₂ is not completely converted in the bioreactor, it leaves the bioreactor in the product gas. The method can be designed in such a way that part of the product gas is fed to the bioreactor. In a preferred embodiment, the method is designed such that the gas mixture exiting the bioreactor, namely the product gas, is fed to a product gas treatment in which at least a portion of the H₂ present in the product gas is separated and returned to the bioreactor. As a result, the CH₄ production rate can be increased, and the H₂ content in the treated product gas can be reduced. In a particularly preferred embodiment, the method is designed such that the separation of the H₂ takes place in a membrane system. In a particularly preferred embodiment, the method is designed such that the separated and recycled H₂-containing gas flow contains a maximum of 90% (v/v) H₂. Limiting the H₂ content is advantageous because it allows the use of simple treatment plants, e.g., single-stage membrane plants, and thus saves energy compared to the separation of almost pure H₂.

In a preferred embodiment, the method is designed such that CO₂ is fed to the bioreactor. In a preferred embodiment, this regulates the pH to below 8.2, significantly contributing to improving process stability. In a preferred embodiment, the method is such that the CO₂ supplied originates from a bioethanol plant. This is advantageous because otherwise the CH₄ production rate from fed H₂ would be limited beyond the amount of CO₂ produced from the organic substrate in the bioreactor. The use of CO₂ from a bioethanol plant is particularly advantageous because this CO₂ was originally captured from the atmosphere during plant growth with the help of sunlight as renewable energy and is produced during fermentation in concentrated form as a gas with over 90% (v/v) CO₂ content. To extract the CO₂ by technical means from the atmosphere or combustion gases instead would cause high energy and operating costs because the CO₂ is present in much lower concentrations, e.g. 0.04% in the atmosphere. In a preferred embodiment, the method is designed such that the product gas is fed to a product gas treatment in which at least a portion of the CO₂ present in the product gas is separated and returned to the bioreactor.

In a preferred embodiment, the method is designed such that the concentration of CO₂ in the product gas does not fall below a value of 5% (v/v), preferably 10% (v/v), particularly preferably 40% (v/v), based on the product gas without the water vapor. In the state of the art, the recommendation is to steer away from such high CO₂ levels, since the goal is usually to reduce the CO₂ content in the product gas as much as possible, preferably to less than 5% (v/v), in order to be able to feed the product gas directly into existing natural gas networks (e.g., EP2771472B1 [0067], EP2586868A2 [0013] and DE102012112889A1 [0041]). Surprisingly, it was found that high CO₂ concentrations can improve the overall effectiveness of the process. Although the cost of gas purification is increased by the higher CO₂ concentrations in the product gas, the advantage of higher bioreactor productivity outweighs this. This enables significantly higher process stability to be achieved in the bioreactor, in particular low pH fluctuations and low concentrations of organic acids. As a result, the bioreactor's organic loading rate capacity is high, which leads to an increase in the CH₄ production rate and an optimal utilization of the available fermentation volume. In a preferred embodiment, the method is designed such that the concentration of CO₂ in the product gas is regulated by the amount of CO₂ supplied to the bioreactor. In a further embodiment, the method is designed such that the regulation of the concentration of CO₂ in the departing product gas is regulated by the amount of H₂ fed to the bioreactor.

In a preferred embodiment, the method is designed in such a way that at least 0.3 mol CO₂, particularly preferably at least 0.5 mol CO₂, is supplied to the bioreactor per mol fed H₂. This has the advantage that the CO₂ content in the product gas is adjusted to a high concentration. The state of the art teaches the opposite, namely no CO₂ at all or no stoichiometric quantity (feeding 0.25 mol CO₂ per mol H₂) to achieve a product gas with the lowest possible CO₂ content. However, the disadvantage of this is process instability.

In a preferred embodiment, the method is designed such that the CO₂ fed to the bioreactor has a purity of at least 90%, particularly preferably 95%. This minimizes the required amount of gas supplied and simplifies the control of the pH value in the bioreactor.

In a preferred embodiment, the method is designed such that the gases supplied to the bioreactor are fed near the bottom of the bioreactor. The advantage of this is the fact that the gas has a longer retention time in the bioreactor volume. In another embodiment, the bioreactors are equipped with stirrers, and gases are fed near the stirrers. The advantage of this is that the contact between gas and liquid is increased.

In a preferred embodiment, the method is designed such that the bioreactor is the first bioreactor in a cascade of bioreactors. This embodiment is particularly advantageous compared to the prior art EP2586868B1, in which the first bioreactor of a bioreactor cascade is not suitable for feeding H₂ due to low process stability. Surprisingly, the invention revealed that high feeding of crude fiber, feeding of low-ammonium liquid, and feeding of CO₂ can address these process stability issues, making the bioreactor volume of the first bioreactor available for the conversion of H₂.

In a preferred embodiment, the method is designed to lower the NH₄—N concentration in the bioreactor. In a preferred embodiment, the method is designed such that the NH₄—N content in the bioreactor is adjusted to a maximum of 6000 mg/kg. This reduction of the NH₄—N content is advantageous, as it also reduces the concentration of ammonia (NH₃) in the bioreactor, which is particularly harmful for process stability. NH₃ is in acid-base equilibrium with ammonium (NH₄⁺) and the proportion of NH₃ increases in proportion to an increasing pH. If the CO₂ feed to the bioreactor is insufficient, the pH in the bioreactor may increase due to the conversion of CO₂ to CH₄, and thus shift the NH₄⁺/NH₃ equilibrium towards the NH₃ with negative consequences. In a further embodiment, the method is designed such that the adjustment of the NH₄—N content is adjusted by selecting the organic substrate fed to the bioreactor. For example, a substrate poor in nitrogen can be selected to be fed to the bioreactor in addition to an organic substrate rich in nitrogen.

In a preferred embodiment, the method is designed such that the NH₄—N content is lowered by the feeding of a low-ammonium liquid, for example, by feeding fresh water. This would result in costly consumption of fresh water. Therefore, in a preferred embodiment, the method is designed such that the low-ammonium liquid is provided by treating the effluent of the bioreactor, in particular by ammonium stripping. Thus, a comparatively low investment and operating cost for effluent treatment avoids the significantly higher costs associated with a fresh water supply.

In a preferred embodiment, the method is designed such that the bioreactor is a stirring tank reactor. In a particularly preferred embodiment, the method is designed such that the bioreactor is operated continuously or semi-continuously. In a preferred embodiment, the method is designed such that the bioreactor does not include any inorganic fillers or growth media.

In a preferred embodiment, there is only a low overpressure of less than 150 mbar in the gas space of the bioreactor. This ensures a cost-saving design, maintenance and a high level of occupational safety. At the same time, the effort required to generate pressure to feed the reactant into the bioreactor and to depressurize it to remove the product gas is minimized.

### BRIEF DESCRIPTION OF THE FIGURES

The invention is explained in more detail below using four embodiments and associated drawings.

FIG. 1, FIG. 2 and FIG. 3 describe the schematic process sequence of some embodiments of the method according to aspects of the invention.

FIGS. 4 (a) and 4 (b) show diagrams of experimental data.

### EMBODIMENT 1

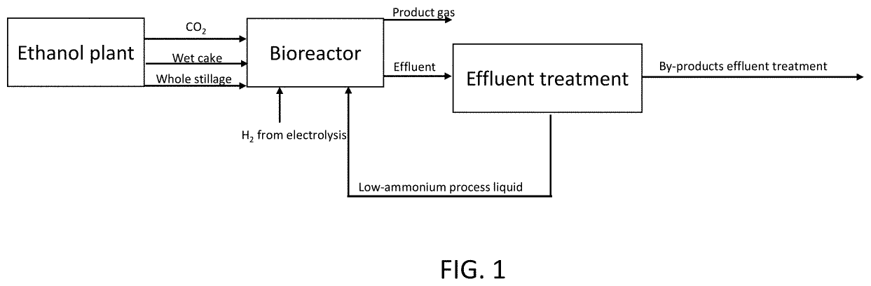

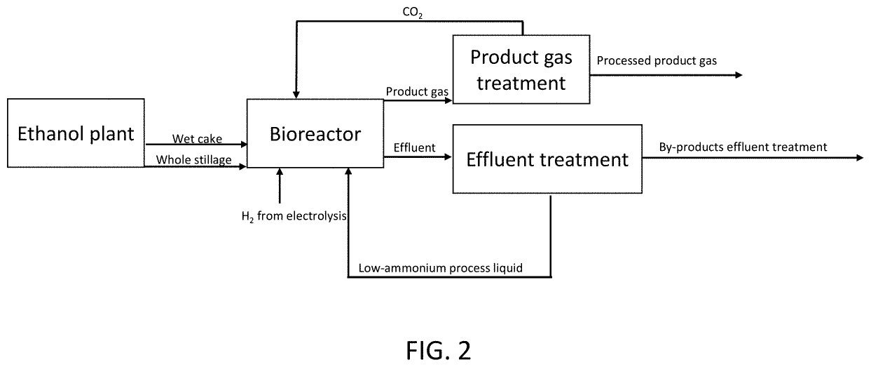

FIG. 1 shows a schematic representation of the method in one embodiment with the feeding of CO₂ from an ethanol plant into the bioreactor as well as an effluent treatment with ammonium stripping and recirculation of the resulting low-ammonium process liquid to adjust the NH₄—N content in the bioreactor. Table 1 shows liquid and solid flows, such as the mass flows of organic substrate in the form of the organic loading rate, crude fiber, and low-ammonium process liquid. Table 2 shows the volume flow rates of the gases fed to and discharged from the bioreactor.

In this and the following examples, the volume flow rates of H₂, CO₂ and H₂S leaving the bioreactor are referred to as H₂, CO₂, and H₂S output rates, respectively, and include the volume of the respective gas under standard conditions (N for short, 273.15 K and 1.01325 bar) in m³ per m³ bioreactor volume discharged from the bioreactor per hour on average. This results in the unit Nm³/m³/h. The term “stillage” refers to the residue from the distillation of a grain mash containing ethanol. The term “whole stillage” is used as a synonym of stillage. In the context of the present invention, “wet cake” refers to the solid phase separated from the stillage by solid-liquid separation.

### Step 1

A daily amount of 11,207 kg of whole stillage from a bioethanol plant is fed as organic substrate to a bioreactor with 1,000 m³ bioreactor volume. The TS content of the whole stillage corresponds to 0.19 kg TS per kg of whole stillage. The VS content of the whole stillage corresponds to 0.91 kg VS per kg TS of whole stillage. This results in a daily feed of whole stillage of 1,937 kg VS or, based on the bioreactor volume of the bioreactor, an organic loading rate of 1.94 kg VS/m³/d.

Based on Weender's food and feed analysis, the crude fiber content of the whole stillage corresponds to 0.039 kg crude fiber per kg TS. Calculated on the basis of the TS content of the whole stillage and the mass flow of whole stillage fed, the result is a very low supply of crude fiber via the whole stillage of only 0.08 kg crude fiber/m³/d.

Surprisingly, it was found that a significantly higher crude fiber content favors the CH₄ production rate from fed H₂, so 4,065 kg/d of wet cake is fed to the bioreactor in addition to the whole stillage. Wet cake contains a high TS content of approx. 0.33 kg TS per kg of wet cake, and an VS content of 0.96 kg VS per kg TS and is typically sold as feed. With a high crude fiber content of 0.14 kg crude fiber per kg TS, wet cake is not a typical organic substrate for biogas production. However, in this embodiment example, it proves to be very useful to significantly increase the crude fiber supply by 0.19 kg crude fiber/m³/d.

Moreover, 6000 kg/d of purified effluent from step 3, 15.8 Nm³ CO₂/h from a bioethanol plant and 25 Nm³ H₂/h from an electrolysis plant are fed to the bioreactor.

TABLE 1
Liquid and solid flows Embodiment 1

| | Numeric | |
|---|---|---|
| | value | Unit |
| Organic loading rate | 1.9 | kg VS/m³/d |
| Whole stillage | | |
| Organic loading rate Wet cake | 1.3 | kg VS/m³/d |
| Crude fiber Whole stillage | 0.08 | kg crude fiber/m³/d |
| Crude fiber Wet cake | 0.19 | kg crude fiber/m³/d |
| Low-ammonium process | 6 | kg/m³/d |
| liquid from step 3 | | |

### Step 2

Organic substrate is converted in the bioreactor to the gases CH₄, CO₂ and H₂S, among others. The H₂ supplied from the electrolysis is converted to approx. 60%. The remaining H₂ leaves the bioreactor with the other gases via the product gas (see Table 2).

The supply of CO₂ from the bioethanol plant results in the CO₂ content in the product gas being approx. 45% (v/v) based on product gas without the water content. Without the CO₂ supply from the bioethanol plant, the CO₂ content in the product gas would only be approx. 37% (v/v) based on product gas without the water content, which would lead to an unwanted, higher pH value. The supply of CO₂ from the bioethanol plant thus results in a more stable reaction in the bioreactor.

The temperature in the bioreactor is controlled at 37° C.

In the embodiment example described here, a CH₄ production rate of 0.053 Nm³/m³/h would be achieved if no H₂ and CO₂ were fed. Using the invention described here, the CH₄ production rate increases to 0.056 Nm³/m³/d by feeding and converting H₂ and CO₂. The CH₄ production rate from fed H₂ corresponds to 0.0038 Nm³/m³/h. This increases the utilization of the available bioreactor volume.

TABLE 2
Gas flows Embodiment 1

| | Numeric | |
|---|---|---|
| | value | Unit |
| Gas feed rates | | |
| H₂ from electrolysis | 0.025 | Nm³/m³/h |
| CO₂ from bioethanol plant | 0.016 | Nm³/m³/h |
| Product gas | | |
| CH₄ production rate | 0.056 | Nm³/m³/h |
| CO₂ output rate | 0.055 | Nm³/m³/h |
| H₂ output rate | 0.010 | Nm³/m³/h |
| H₂S output rate | 0.001 | Nm³/m³/h |

### Step 3

The effluent of the bioreactor is fed into an effluent treatment using ammonium stripping, and ammonium is largely removed. The resulting process liquid, which is low in ammonium, has an NH₄—N content of 500 mg/kg. A part of this low-ammonium process liquid is fed to the bioreactor to adjust the NH₄—N content, thereby adjusting the NH₄—N content to below 6000 mg/kg.

### EMBODIMENT 2

Another option for process control consists of utilizing the CO₂ produced from organic substrate during biogas production instead of CO₂ from a bioethanol plant. For this purpose, a product gas treatment is added to the method described in embodiment 1, in which CO₂ is separated from the product gas at an hourly circulation rate of 0.016 Nm³ CO₂ per m³ bioreactor volume and fed back into the bioreactor. This CO₂ replaces the CO₂ flow from the bioethanol plant described in embodiment 1. FIG. 2 shows a schematic illustration of this method. Table 3 shows the gas flows. The liquid flows in this example are the same as in embodiment 1 and shown in Table 1. The temperature in the bioreactor is still controlled at 37° C. and the effluent treatment from step 3 in embodiment 1 is maintained.

In this context and as used hereinafter, circulation rate refers to the volume of the respective gas under standard conditions (N for short, 273.15 K and 1.01325 bar) in m³ per m³ bioreactor volume, which is partially separated from the product gas in the product gas treatment and fed back into the bioreactor on average per hour. This results in the unit Nm³/m³/h.

As in embodiment 1, the CO₂ content in the product gas is 45% (v/v) based on product gas without the water component and thus advantageously higher than it would be at 37% (v/v) based on product gas without the water component and if CO₂ had not been fed.

Since the changed CO₂ source has no effect on the CH₄ production rate with otherwise constant operating conditions as in embodiment 1, the CH₄ production rate based on the supplied H₂ is also 0.0038 Nm³/m³/h.

TABLE 3
Gas flows Embodiment 2

| | Numeric | |
|---|---|---|
| | value | Unit |
| Gas feed rates | | |
| H₂ from electrolysis | 0.025 | Nm³/m³/h |
| CO₂ from product gas treatment | 0.016 | Nm³/m³/h |
| Product gas | | |
| CH₄ | 0.056 | Nm³/m³/h |
| CO₂ output rate | 0.055 | Nm³/m³/h |
| H₂ output rate | 0.010 | Nm³/m³/h |
| H₂S output rate | 0.001 | Nm³/m³/h |
| Product gas treatment | | |
| CO₂ circulation rate | 0.016 | Nm³/m³/h |
| Processed product gas | | |
| CH₄ production rate | 0.056 | Nm³/m³/h |
| CO₂ | 0.039 | Nm³/m³/h |
| H₂ | 0.010 | Nm³/m³/h |
| H₂S | 0.001 | Nm³/m³/h |

### EMBODIMENT 3

If feeding product gas into the natural gas network is planned, certain rules stipulated by the network operator regarding the composition of the gas must be observed. As a rule, for example, the CH₄ content must be greater than 95% (v/v), the H₂ content must be less than 2% (v/v) and the H₂S content must be approx. 0% (v/v) based on gas without the water content.

FIG. 3 shows a schematic representation of a modified method according to embodiment 2 with upgraded product gas treatment and gas recirculation into the bioreactor. H₂S is almost completely removed from the product gas. A large part of the CO₂ is removed from the product gas and some of it is returned to the bioreactor, while the rest is sent for other utilization. H₂ is separated via a membrane. In terms of investment and operating costs, the fact that only one separation stage is used proves to be advantageous. This is sufficient to maintain a typical H₂ maximum concentration of 2% (v/v) for gas injection into a natural gas grid, but results in some CH₄ being returned to the bioreactor along with the H_{2.}

Table 4 shows the gas flows in this embodiment. The liquid flows in this example are the same as in embodiment 1 and shown in Table 1. The temperature in the bioreactor is still controlled at 37° C. and the effluent treatment from step 3 in embodiment 1 is maintained.

Recirculation of H₂ increases the retention time of H₂ in the bioreactor. This allows the conversion of the H₂ coming from the electrolyzer to be increased from 60% to 95% (v/v). The CH₄ production rate from supplied H₂ thus corresponds to 0.00594 Nm³/m³/h. Due to the increased amounts of recycled gas, the increase in recycled CO₂ is also necessary to keep the CO₂ content in the product gas high. Thus, the advantages of a stable CO₂ concentration in the product gas described in embodiment 1 can be maintained.

TABLE 4
Gas flows Embodiment 3

| | Numeric | |
|---|---|---|
| | value | Unit |
| Gas feed rates | | |
| H₂ feed rate (from electrolysis) | 0.025 | Nm³/m³/h |
| CO₂ from product gas treatment | 0.028 | Nm³/m³/h |
| H₂ feed rate (from product gas | 0.015 | Nm³/m³/h |
| treatment) | | |
| CH₄ from product gas treatment | 0.004 | Nm³/m³/h |
| Product gas | | |
| CH₄ | 0.063 | Nm³/m³/h |
| CO₂ output rate | 0.065 | Nm³/m³/h |
| H₂ output rate | 0.016 | Nm³/m³/h |
| H₂S output rate | 0.001 | Nm³/m³/h |
| Product gas treatment | | |
| CO₂ circulation rate | 0.028 | Nm³/m³/h |
| CO₂ for other utilization | 0.034 | Nm³/m³/h |
| H₂ circulation rate | 0.015 | Nm³/m³/h |
| CH₄ circulation rate | 0.004 | Nm³/m³/h |
| H₂S | 0.001 | Nm³/m³/h |
| Processed product gas | | |
| CH₄ | 0.058 | Nm³/m³/h |
| production rate | | |
| CO₂ | 0.002 | Nm³/m³/h |
| H₂ | 0.001 | Nm³/m³/h |
| H₂S | 0.000 | Nm³/m³/h |

### EMBODIMENT 4

Embodiment 4 includes experiments on a pilot-plant scale.

### Method

Two bioreactors “A” and “B” of the stirring-tank-reactor type were fed with different organic substrates as well as H₂ and CO₂.

Bioreactor A was fed with whole stillage and low-ammonium process liquid from a bioethanol plant. Feeding was carried out semi-continuously, distributed in five equal intervals per day.

Bioreactor B was fed with cereal straw and low-ammonium process liquid. Feeding was done once a day.

The bioreactor volume corresponded to 75 l (bioreactor A) and 55 l (bioreactor B). Mixing was performed with central stirrers at a rotational speed of 370 rpm for both bioreactors. The temperature of the bioreactor volume was controlled using water as a heating medium at about 39° C., which flowed around the bioreactors via a double casing. The supplied quantities of H₂ and CO₂ were controlled separately for each gas via thermal mass flow controllers (EL-Flow Select, Bronkhorst Deutschland Nord GmbH). Product gas volume flows were determined in both bioreactors using drum gas meters (TG 0.5, Dr.-Ing. RITTER Apparatebau GmbH & Co. KG). The gas composition of the product gas was determined once a week using gas chromatography (MobilGC, ECH Elektrochemie Halle GmbH; configuration: Hayesep QS column 45° C., molecular sieve column 55° C., thermal conductivity detector, carrier gas argon). The TS content of the substrates was determined gravimetrically after drying at 105° C. until mass constancy was achieved. The ash content was determined gravimetrically after annealing at 650° C. for at least two hours. The VS content was calculated based on the difference between the dry substance content and the ash content. The crude fiber content was determined according to the VDLUFA method book (see section Definitions). The low-ammonium process liquid was neglected in the determination of the organic loading rate and the fed crude fiber.

### Results and Discussion

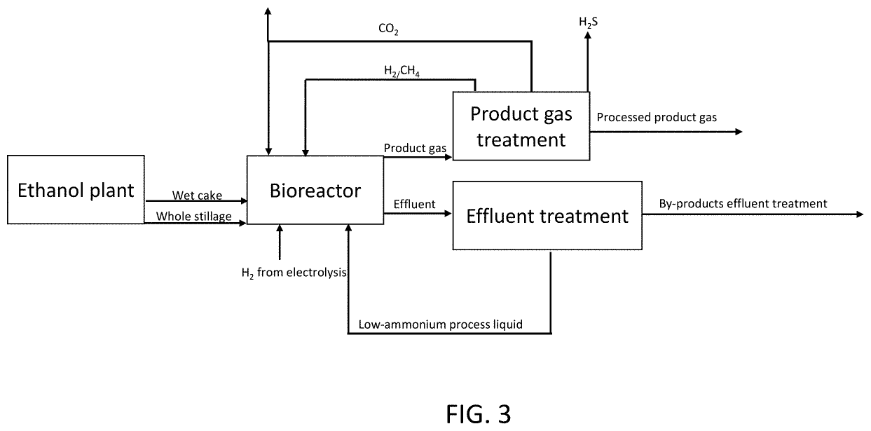

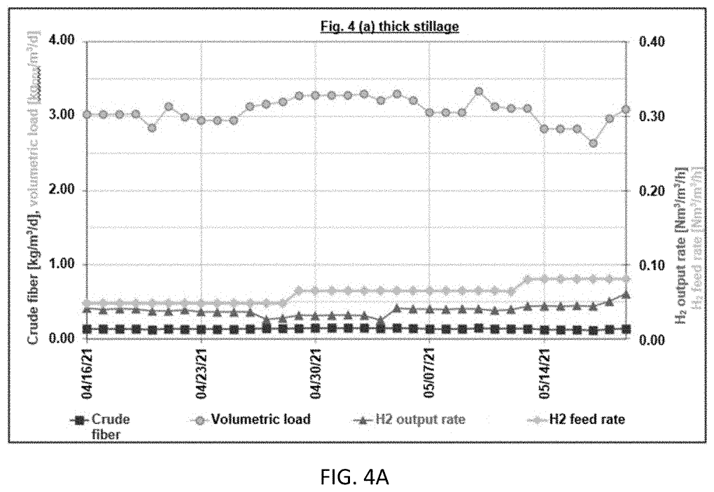

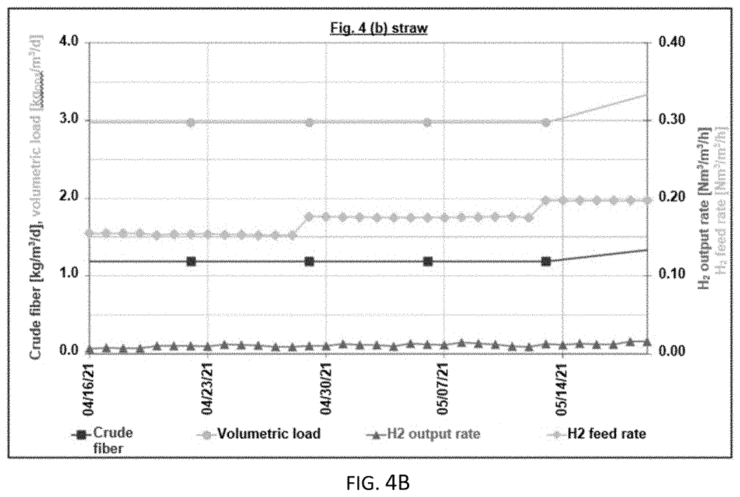

FIG. 4 shows data from the two bioreactors dated Apr. 16, 2021 to May 19, 2021. Prior to this, both reactors had already been operated for more than one year, hence transient effects of the start-up phase can be excluded and neglected in the interpretation of the data.

In both bioreactors, organic substrate is converted to biogas by means of microorganisms, as indicated by a lower amount of VS in the effluent compared to the reactor feed, as well as by means of the measured CH₄ production rates, which are higher than the calculated CH₄ production rates from fed H₂ and therefore must originate from organic substrate decomposition (data not shown).

The organic loading rate is comparable for both reactors at approx. 3 kg VS/m³/d. However, the feeding of crude fiber differs significantly. While in bioreactor A only about 0.14 kg crude fiber/m³/d was fed via the whole stillage, the amount in bioreactor B via the straw was significantly higher at about 1.2 kg crude fiber/m³/d.

Bioreactor B exhibited significantly higher performance for in-situ methanation of H₂ and CO₂. Even at high H₂ feed rates of 0.197 Nm³/m³/h, low H₂ output rates of about 0.014 Nm³/m³/h were measured. Thus, according to equation (2), the CH₄ production rate from fed H₂ is about 0.046 Nm³/m³/h. In bioreactor A, on the other hand, higher H₂ output rates were measured than in bioreactor B of 0.048 Nm³/m³/h, despite significantly lower H₂ feed rates of only 0.081 Nm³/m³/h. This corresponds to a CH₄ production rate from fed H₂ in bioreactor A of 0.008 Nm³/m³/h.

### LIST OF REFERENCES

Schiraldi C., De Rosa M. (2014) Mesophilic Organisms. In: Drioli E., Giorno L. (eds) Encyclopedia of Membranes. Springer, Berlin, Heidelberg. https://doi.org/10.1007/978-3-642-40872-4_1610-2

1. A method for the biological in-situ methanation of carbon dioxide and hydrogen in a bioreactor containing microorganisms and having a bioreactor volume, the method comprising:
a) feeding an organic substrate to the microorganisms in the bioreactor to produce a biogas comprising methane and carbon dioxide, wherein the organic substrate includes crude fiber and organic dry substance (VS), and wherein the organic substrate fed to the bioreactor includes at least 0.15 kg of crude fiber per m³ of the bioreactor volume per day;
b) operating the bioreactor at between 20-45° C.;
c) feeding hydrogen into the bioreactor, wherein and at least part of the hydrogen together with the carbon dioxide is converted to methane by the microorganisms; and
d) removing a product gas from the bioreactor, wherein the product gas includes methane.

2. The method according to claim 1 wherein the product gas further comprises carbon dioxide, hydrogen, and water vapor.

3. The method according to claim 1, wherein the bioreactor is operated at an organic loading rate of at least 2.5 kg VS per m³ of the bioreactor volume per day.

4. The method according to claim 1, wherein the bioreactor is operated at an organic loading rate of at least 3.0 kg VS per m³ of the bioreactor volume per day.

5. The method according to claim 1, wherein at least 0.2 kg crude fiber per m³ of the bioreactor volume per day is fed to the bioreactor,

6. The method according to claim 1, wherein at least 1.0 kg crude fiber per m³ of the bioreactor volume per day is fed to the bioreactor.

7. The method according to claim 1, wherein the hydrogen is fed into the bioreactor at a rate of at least 0.017 Nm³ per m³ of the bioreactor volume per hour.

8. The method according to claim 1, wherein the hydrogen is fed into the bioreactor at a rate of at least 0.125 Nm³ per m³ of the bioreactor volume per hour.

9. The method according to claim 1, further comprising feeding carbon dioxide to the bioreactor.

10. The method according to claim 9, further comprising adjusting the amount of carbon dioxide fed to the bioreactor such that the concentration of carbon dioxide in the product gas without the water vapor does not fall below a value of 5% (v/v).

11. The method according to claim 9, further comprising adjusting the amount of carbon dioxide fed to the bioreactor such that the concentration of carbon dioxide in the product gas without the water vapor does not fall below a value of 40% (v/v).

12. The method according to claim 9, further comprising regulating the concentration of carbon dioxide in the product gas by adjusting the amount of carbon dioxide fed to the bioreactor.

13. The method according to claim 9, further comprising regulating the concentration of carbon dioxide in the product gas by adjusting the amount of hydrogen fed to the bioreactor.

14. The method according to claim 9, further comprising feeding the carbon dioxide in an amount which adjusts the pH in the bioreactor to below 8.2.

15. The method according to claim 1, wherein the bioreactor has a NH₄—N content and the method further comprises adjusting the NH₄—N content of the bioreactor to levels below 6,000 mg/kg by feeding a low ammonium liquid and/or by the selection of the organic substrate.

16. The method according to claim 15, wherein the low-ammonium liquid is effluent from the bioreactor whose NH₄—N content has been reduced via ammonium stripping.

17. The method according to claim 2, further comprising separating hydrogen from the product gas and returning the separated hydrogen to the bioreactor.

18. The method according to claim 2, further comprising separating carbon dioxide from the product gas and returning at least a portion of the separated carbon dioxide to the bioreactor.

19. A method for the biological in-situ methanation of carbon dioxide and hydrogen in a bioreactor containing microorganisms and having a bioreactor volume, the method comprising:
a) feeding an organic substrate to the microorganisms in the bioreactor to produce a biogas comprising methane and carbon dioxide;
b) operating the bioreactor at between 20-45° C.;
c) feeding hydrogen to the carbon dioxide in the bioreactor to produce methane;
d) removing a product gas from the bioreactor, wherein the product gas includes methane, carbon dioxide, and hydrogen;
e) separating hydrogen from the product gas and returning the separated hydrogen to the bioreactor; and
f) separating carbon dioxide from the product gas and returning at least a portion of the separated carbon dioxide to the bioreactor.

20. A method for the biological in-situ methanation of carbon dioxide and hydrogen in a bioreactor containing microorganisms and having a bioreactor volume, the method comprising:
a) feeding an organic substrate to the microorganisms in the bioreactor to produce a biogas comprising methane and carbon dioxide, wherein the organic substrate includes crude fiber, and wherein the organic substrate fed to the bioreactor includes at least 0.15 kg of crude fiber per m³ of the bioreactor volume per day;
b) operating the bioreactor at between 20-45° C.;
c) feeding hydrogen to the carbon dioxide in the bioreactor to produce methane;
d) feeding carbon dioxide into the bioreactor;
e) removing a product gas from the bioreactor, wherein the product gas includes methane produced in step (a) and step (c), carbon dioxide, and hydrogen;
f) adjusting the amount of carbon dioxide fed to the bioreactor such that the concentration of carbon dioxide in the product gas without the water vapor does not fall below a value of 10% (v/v).
